(19) United States
(12) Patent Application Publication
(10) Pub. No.: US 2007/0294037 A1
(43) Pub. Date: Dec. 20, 2007

(54) System and Method for Optimizing Data Acquisition of Plasma Using a Feedback Control Module

(76) Inventors: Sang Hun Lee, San Ramon, CA (US); Jay Joongsoo Kim, Los Altos, CA (US)

Correspondence Address: BUCHANAN, INGERSOLL & ROONEY PC, POST OFFICE BOX 1404, ALEXANDRIA, VA, 22313-1404, US

(21) Appl. No.: 11/661,067

(22) PCT Filed: Aug. 2, 2005
(86) PCT No.: PCT/US05/27476

§ 371 Date: Feb. 22, 2007

Related U.S. Application Data
(63) Continuation of application No. 10/935,743, filed on Sep. 8, 2004.

(51) Int. Cl. G01N 33/48

(52) U.S. Cl. 702/19

(57) ABSTRACT

Method, systems and computer readable media for optimizing data acquisition of microwave plasma are disclosed. The present invention provides a method that includes the steps of selecting an operational condition for a plasma generation system, operating the plasma generation system under the selected operational condition, determining whether a stable plasma is established using a sensing device and acquiring/storing plasma data if the stable plasma is established. The method further includes a step of repeating data acquisition under various operational conditions to establish a database for plasma characterization. The present invention further provides a feedback control module that operates in conjunction with a plasma generating system to automate and optimize the process of data acquisition.

### BACKGROUND OF THE INVENTION

1. Field of the Invention

The present invention relates to data acquisition systems, and more particularly to systems and methods for optimizing data acquisition using a feedback control module.

2. Discussion of the Related Art

In recent years, the progress on producing plasma has been increasing. Typically, plasma consists of positive charged ions, neutral species and electrons. In general, plasmas may be subdivided into two categories: thermal equilibrium and thermal non-equilibrium plasmas. Thermal equilibrium implies that the temperature of all species including positive charged ions, neutral species, and electrons, is the same.

Plasmas may also be classified into local thermal equilibrium (LTE) and non-LTE plasmas, where this subdivision is typically related to the pressure of the plasmas. The term “local thermal equilibrium (LTE)” refers to a thermodynamic state where the temperatures of all of the plasma species are the same in the localized areas in the plasma.

A high plasma pressure induces a large number of collisions per unit time interval in the plasma, leading to sufficient energy exchange between the species comprising the plasma, and this leads to an equal temperature for the plasma species. A low plasma pressure, on the other hand, may yield one or more temperatures for the plasma species due to insufficient collisions between the species of the plasma.

In non-LTE, or simply non-thermal plasmas, the temperature of the ions and the neutral species is usually less than 100° C., while the temperature of electrons can be up to several tens of thousand degrees in Celsius. Therefore, non-LTE plasma may serve as highly reactive tools for powerful and also gentle applications without consuming a large amount of energy. This “hot coolness” allows a variety of processing possibilities and economic opportunities for various applications. Powerful applications include metal deposition systems and plasma cutters, and gentle applications include plasma surface cleaning systems and plasma displays.

One of these applications is plasma sterilization, which uses plasma to destroy microbial life, including highly resistant bacterial endospores. Sterilization is a critical step in ensuring the safety of medical and dental devices, materials, and fabrics for final use. Existing sterilization methods used in hospitals and industries include autoclaving, ethylene oxide gas (EtO), dry heat, and irradiation by gamma rays or electron beams. These technologies have a number of problems that must be dealt with and overcome and these include issues such as thermal sensitivity and destruction by heat, the formation of toxic byproducts, the high cost of operation, and the inefficiencies in the overall cycle duration. Consequently, healthcare agencies and industries have long needed a sterilizing technique that could function near room temperature and with much shorter times without inducing structural damage to a wide range of medical materials including various heat sensitive electronic components and equipment.

These changes to new medical materials and devices have made sterilization very challenging using traditional sterilization methods. One approach has been using a low pressure plasma (or equivalently, a below-atmospheric pressure plasma) generated from hydrogen peroxide. However, due to the complexity and the high operational costs of the batch process units needed for this process, hospitals use of this technique has been limited to very specific applications. Also, low pressure plasma systems generate plasmas having radicals that are mostly responsible for detoxification and partial sterilization, and this has negative effects on the operational efficiency of the process.

As opposed to low pressure plasmas associated with vacuum chambers, atmospheric pressure plasmas for sterilization, as in the case of material processing, offer a number of distinct advantages to users. Its compact packaging makes it easily configurable, it eliminates the need for highly priced vacuum chambers and pumping systems, it can be installed in a variety of environments without additional facilitation needs, and its operating costs and maintenance requirements are minimal. In fact, the fundamental importance of atmospheric plasma sterilization lies in its ability to sterilize heat-sensitive objects, its simple-to-use, and has a faster turnaround cycle. Atmospheric plasma sterilization may be achieved by the direct effect of reactive neutrals, including atomic oxygen and hydroxyl radicals, and plasma generated UV light, all of which can attack and inflict damage to bacteria cell membranes.

One of the essential procedures for developing non-LTE plasma systems may be characterizing the thermo-physical and/or thermo-chemical properties of non-LTE plasmas, such as plasma electron density, electron temperature, neutral species temperature and species concentration under various operational conditions. Typically, a plasma characterization may require a database that may include data of a considerable size, such as high resolution plasma image, emission spectra, etc., taken under each operational condition. Establishing a database for the plasma characterization may have challenging problems to overcome. Firstly, development engineers may perform measurements under potentially hazardous operating conditions as the engineers may operate the system without knowing the operational characteristics of the system under the development. This safety issue becomes more pronounced for atmospheric pressure plasma measurements since development engineers may be exposed directly to the plasma as well as the heating source, such as microwaves or RF. Secondly, the engineers may have to acquire the data under various operational conditions during the development stage. Such data acquisition process may be tedious and prone to human errors. Thus, there is a need for a system that may provide safe, efficient and reliable ways to acquire the data for a plasma generating system.

### SUMMARY OF THE INVENTION

The present invention provides a feedback control module that operates in conjunction with a plasma generating system to optimize data acquisition. The feedback control module may operate one or more components of the plasma generating system in accordance with predetermined operational conditions. For each operational condition, the feedback control module may determine whether a stable plasma is established. To optimize the data acquisition, the feedback control module may communicate with measurement devices to obtain and store the data only if the stable plasma is established. Also, the entire operation of the feedback control module may be automated so that the data acquisition may be performed without introducing any human error or a potential injury.

According to one aspect of the present invention, a system for acquiring plasma data comprises: a microwave generator for generating microwaves; a power supply connected to the microwave generator for providing power thereto; a microwave cavity having a wall forming a portion of a gas flow passage; a waveguide operatively connected to the microwave cavity for transmitting microwaves thereto; a coupler operatively connected to the waveguides; a power meter, connected to the coupler, for measuring microwave fluxes; an isolator, operatively connected to the waveguide, for dissipating microwaves reflected from the microwave cavity; a gas flow control mechanism coupled to the gas flow passage of the microwave cavity for controlling a gas flow rate; a nozzle operatively coupled to the gas flow passage of the microwave cavity and configured to generate plasma from a gas flow and microwaves received from the microwave cavity; a sensing device configured to respond to a characteristic quantity of the plasma; at least one measurement device configured to acquire data; and a feedback control module connected to the power supply, the power meter, the sensing device, the at least one measurement device and the gas flow control, the feedback control module being configured to control the power supply, the at least one measurement device and the gas flow control and to receive at least one signal from the power meter and the sensing device.

According to another aspect of the present invention, a computer including a processor for running a computer-readable program code in a memory comprises: a recipe file having at least one recipe that specifies at least one operational condition of a plasma generating system; a feedback control manager structured and arranged to control the plasma generating system under the at least one operational condition, the feedback control manager comprising: a recipe interpreter for interpreting the at least one recipe; and a recipe sequencer for sequencing the recipe into at least one command to control the plasma generating system; a data acquisition manager configured to acquire data if the plasma generating system generates a stable plasma under the at least one operational condition; and an open database connectivity configured to store the data.

According to yet another aspect of the present invention, a method for acquiring plasma data comprises the steps of: selecting an operational condition for a plasma generation system; operating the plasma generation system under the operational condition selected in the step of selecting; determining whether a stable plasma is established using a sensing device; evaluating whether a stable plasma is determined in the step of determining, if so then the method includes the steps of: acquiring data, and storing the data obtained in the step of acquiring; determining whether an additional measurement is needed, wherein, if the additional measurement is not needed, the method further comprises the step of terminating data acquisition process; changing the operational condition selected in the step of selecting; and repeating the above steps for a new operational condition determined in the step of changing.

According to still another aspect of the present invention, a feedback control module for acquiring data of a plasma generated by a gas flow heated by microwaves comprises: a first field Input/Output coupled to a power control configured to control microwave generation; a universal serial bus/general-purpose interface bus (QBS/GPIB) converter coupled to a power meter that is configured to measure fluxes of the microwaves; a second field Input/Output coupled to a sensing device that is configured to generate a signal in response to a characteristic quantity of plasma; a third field Input/Output coupled to a measurement device that is configured to acquire plasma data if a stable plasma is established; a fourth field Input/Output coupled to a gas flow control; and a computer having an interface coupled to the first, second, third and fourth field Input/Outputs and the USB/GPIB converter, and the interface comprising a plurality of interface components.

These and other advantages and features of the invention will become apparent to those persons skilled in the art upon reading the details of the invention as more fully described below.

### BRIEF DESCRIPTION OF THE DRAWINGS

FIG. 1 is a schematic diagram of a plasma generating system that is coupled with a feedback control/data acquisition system in accordance with one embodiment of the present invention.

FIG. 2 is a detailed schematic diagram of the feedback control module shown in FIG. 1.

FIG. 3 shows a schematic diagram of an exemplary computer that may be used in embodiments of the present invention.

FIG. 4 schematically illustrates the architecture of the software components that relate to the feedback control/data acquisition in accordance with one embodiment of the present invention.

FIG. 5 is a flowchart illustrating the exemplary steps of the feedback control module shown in FIG. 1 in accordance with one embodiment of the present invention.

FIG. 6 is a block diagram illustrating the steps for converting a recipe into a sequence of commands taken by a feedback control manager in accordance with one embodiment of the present invention.

FIG. 7 is a partial cross-sectional view of the microwave cavity and nozzle shown in FIG. 1.

FIG. 8 is an exploded view of the components comprising the nozzle shown in FIG. 7.

### DETAILED DESCRIPTION OF THE PREFERRED EMBODIMENTS

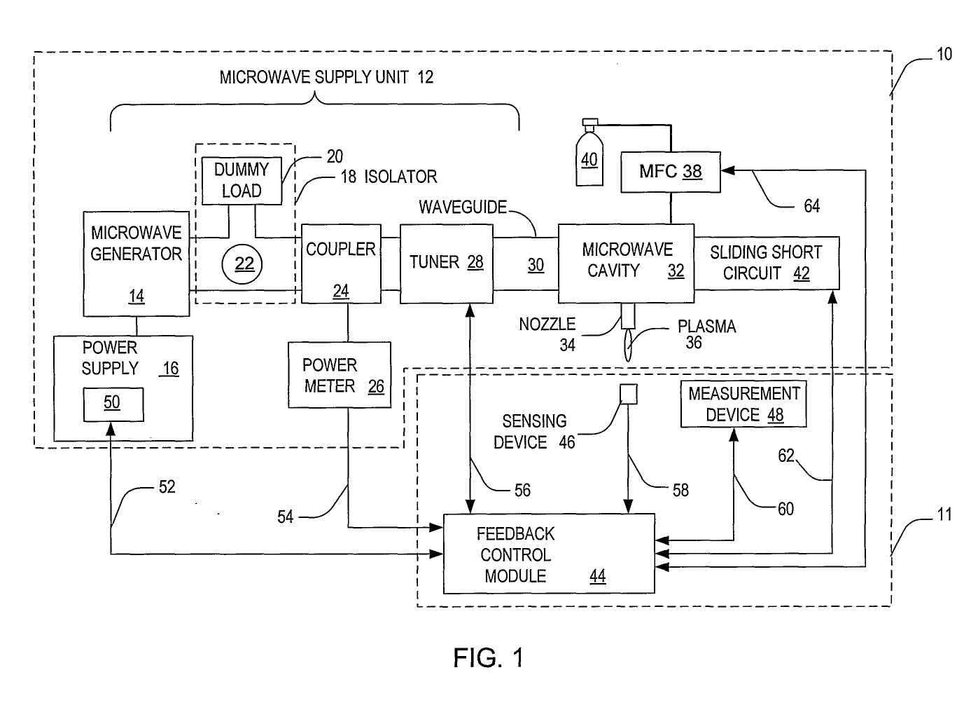

FIG. 1 is a schematic diagram of a plasma generating system 10 coupled with a feedback control module/data acquisition system 11 in accordance with one embodiment of the present invention. As illustrated, the plasma generating system 10 comprises: a microwave supply unit 12 for generating microwaves; a microwave cavity 32; a waveguide 30 for transmitting microwaves from the microwave supply unit 12 to the microwave cavity 32; and a nozzle 34, connected to the microwave cavity 32, for receiving microwaves from the microwave cavity 32 and generating a plasma 36 from a gas and/or gas mixture received from a gas tank 40 via a gas flow control that is preferably, but not limited to, a Mass Flow Control (NgC) valve 38. In one embodiment, a sliding short circuit 42 may be attached to the microwave cavity 32 and it controls the microwave energy distribution within the microwave cavity 34 by adjusting the microwave phase.

The microwave supply unit 12 provides microwaves to the microwave cavity 32 and may include: a microwave generator 14 for generating microwaves; a power supply 16 for supplying power to the microwave generator 14, the power supply 16 having a power control 50 that controls the power level of the power supply 16; and an isolator 18 having a dummy load 20 for dissipating the retrogressing microwaves that propagate toward the microwave generator 14 and a circulator 22 for diverting the retrogressing microwaves to the dummy load 20. The microwave supply unit 12 further includes: a coupler 24 for coupling fluxes of microwaves and a power meter 26 connected to the coupler 24 for measuring the fluxes of the microwaves. In one embodiment, the microwave supply unit 12 may include a tuner 28 for matching the impedance.

The components of the plasma generating system 10 are provided for exemplary purposes only. Thus, it should be apparent to one of ordinary skill that a system with a capability to provide plasma may replace the plasma generating system 10 without deviating from the present invention. For example, various plasma generating systems are described in PCT Application entitled “Microwave Plasma Nozzle with Enhanced Plume Stability and Heating Efficiency” filed on Jul. 7, 2005, PCT Application entitled “System and Method for Controlling A Power Distribution Within a Microwave Cavity” filed Jul. 21, 2005 and PCT application entitled “Plasma Nozzle Array for Providing Uniform Scalable Microwave Plasma Generation” filed Jul. 21, 2005, which are incorporated herein by reference.

Still referring to FIG. 1, the feedback control module/data acquisition system 11 may include: a feedback control module 44; a sensing device 46 and a measurement device 48 for obtaining a data related to, but not limited to, plasma characterization. The sensing device 46 may be any type of sensing device that can convert the intensity of plasma radiation into an electric signal, such as photodiode, UV detector, phototransistor, photocell or photoconductive cell, where the electric signal level may be proportional to the intensity of plasma radiation. As will be explained later, the main function of the sensing device 46 may be converting the intensity of a characteristic quantity of the plasma 36 into an electric signal. Thus, it should be apparent to those of ordinary skill in the art that other conventional types of devices, such as a thermocouple, may be used as a sensing device, where the thermocouple detects the temperature of the neutral species of the plasma 36. Likewise, the type of the measurement device 48 may be determined by the designer and/or the operator of the feedback control module/data acquisition system 11. For example, an optical spectrograph may be used to capture the plasma spectra. In another example, a digital camera may be used to take high-resolution plasma images. In still another example, the measurement device 48 may comprise more than one device.

As illustrated in FIG. 1, the feedback control module 44 may be coupled to various components of the plasma generating system 10. Details of the interaction between the feedback control module 44 and these components will be given in following sections.

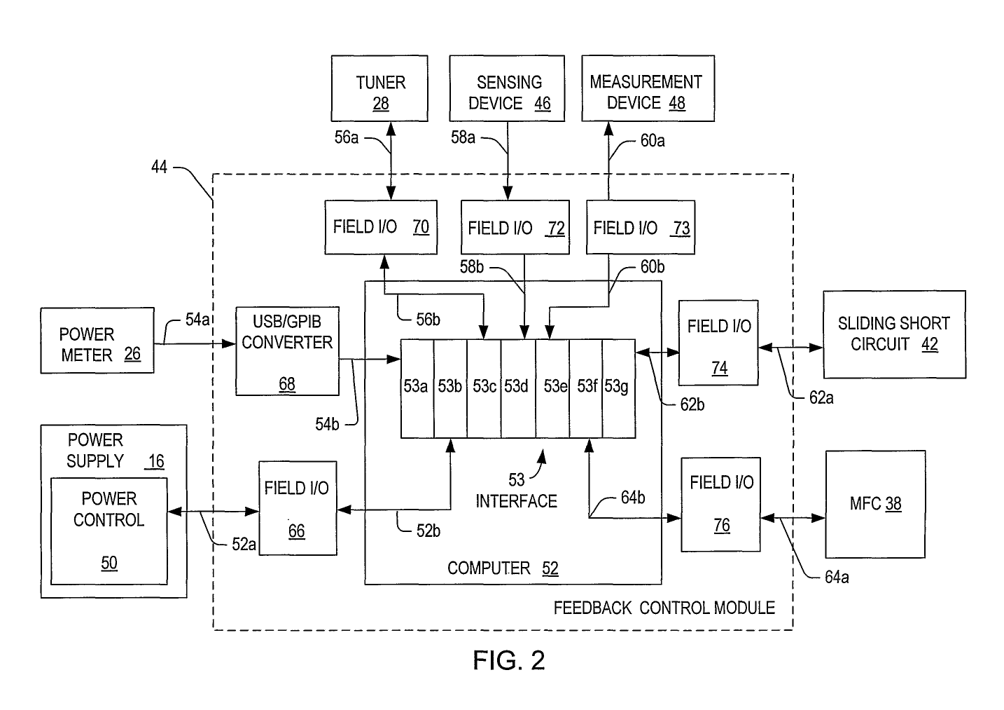

FIG. 2 is a detailed schematic diagram of the feedback control module 44 shown in FIG. 1. The feedback control module 44 includes a computer 52 having an interface 53 for communicating with various components of the plasma generating system 10, wherein the interface 53 includes seven components, 53a-53g. Each of the components 53a-53g may be an interface device, such as a DeviceNet™ network, a Profinet™ network, an Ethernet/IP™ network and an Opto22™ network, and is adapted to communicate with a corresponding component of the plasma generating system 10 in a pre-selected commercial industrial protocol (CIP). The interface component 53 may be connected to USB/GPIB converter 68 that is coupled to the power meter 26 through lines 54a and 54b, a field Input/Output (I/O) 66 coupled to the power control 50 through lines 52a and 52b, a field I/O 70 coupled to the tuner 28 through lines 56a and 56b, a field I/O 72 connected to a sensing device 46 through lines 58a and 58b, a field I/O 73 connected to the measurement device 48 through lines 60a and 60b, a field I/O 76 coupled to the MFC valve 38 through lines 64a and 64b, and a field I/O 74 coupled to the sliding short circuit 42 through lines 62a and 62b, respectively. In FIG. 2, the field I/Os 68, 70, 72, 73, 74 and 76 are illustrated as individual components of the feedback control module 44. However, it should be apparent to those of ordinary skill in the art that they may be implemented in one or more field I/O modules without deviating from the present invention. Also, depending on the type of a plasma generating system, the present invention may be practiced with other types and different numbers of interface components than those of the interface 53.

As illustrated in FIG. 2, lines 52a and 52b, 54a and 54b, 56a and 56b, 58a and 58b, 60a and 60b, 62a and 62b and 64a and 64b may transmit control signals (or, equivalently “output signals”) from the feedback control module 44 and/or status signals (or, equivalently “input signals”) from the components, where each status signal indicates the setpoint of its corresponding component. Each of the lines 52a and 52b may comprise four signal lines for operating the power control 50: an output line (preferably in analog format) for controlling power level; an input line (preferably in analog format) for power level status, a digital output (DO) line for controlling the ON/OFF switching of the power control 50 and a digital input (DI) line for the ON/OFF status of the power control 50. Each of the lines 54a and 54b may transmit an input signal for the status of the microwave flux measured by the power meter 26 to a feedback control module 44, where the USB/GPIB converter may convert the input signal sent from a GPIB of the power meter 26 to a compatible USB signal. Each of the lines 56a and 56b may comprise two signal lines for controlling the tuner 28; output and input signal lines for controlling a tuner configuration and obtaining the status of the tuner configuration, respectively. Each of the lines 58a and 58b may transmit an input signal from the sensing device 46, where the input signal may be used to determine whether stable plasma is established, as will be explained later. Each of the lines 60a and 60b may transmit an output signal to control the measurement device 48 for data acquisition and the input signal that contains the acquired data. The types of the lines 60a and 60b and the field I/O 73 may be dependent on that of the measurement device 48. For example, an RS232 line may be used for spectral measurement devices without the field I/O 73. Each of the lines 62a and 62b may comprise two or more signal lines for controlling a sliding short circuit 28; output and input signal lines for controlling the position of a slider contained in a sliding short circuit 28 and obtaining the status of a slider position, respectively. Each of the lines 64a and 64b may comprise four or more signal lines for controlling a gas flow rate through the MFC valve 38: an output line for controlling the MFC valve 38 to adjust gas flow rate; an input line for the status of the MFC valve switching 38, a digital output (DO) line for controlling the ON/OFF of the MFC valve 38 and a digital input (DI) line for the ON/OFF status of the MFC valve 38. The number of signal lines consisting each of lines 52a and 52b, 54a and 54b, 56a and 56b, 58a and 58b, 60a and 60b, 62a and 62b and 64a and 64b may change depending on the type and/or model of the corresponding component. Thus it should be apparent to those of ordinary skill that the number of signal lines and types of signals set forth above are for exemplary purposes and may change without diverting from the present invention.

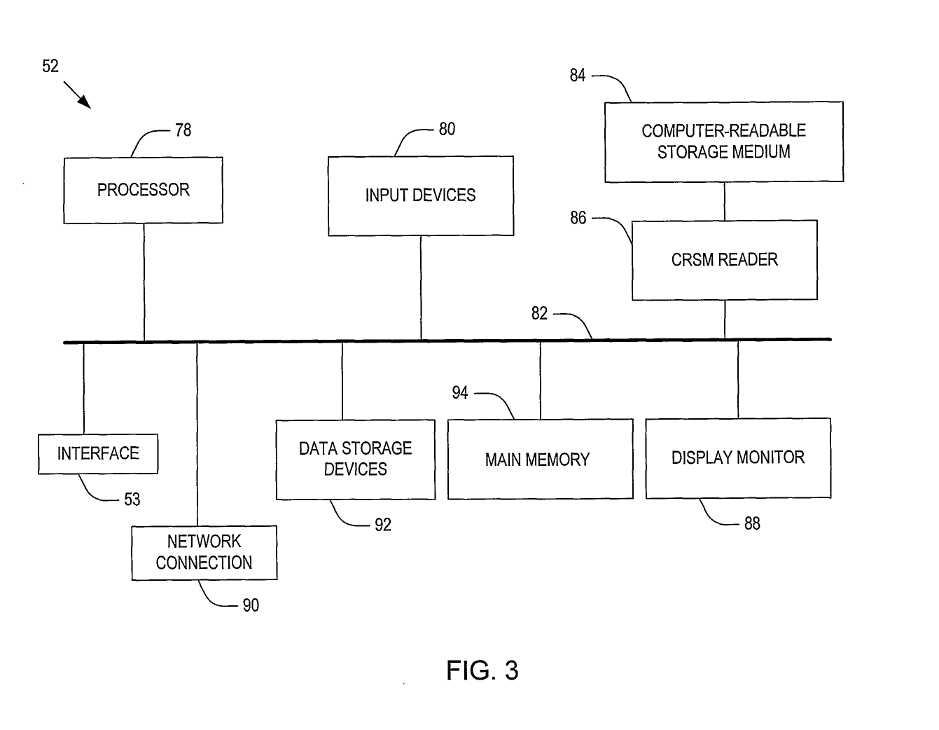

FIG. 3 is a schematic diagram of an example computer 52 that may be used in the embodiments of the present invention. Being computer-related, it can be appreciated that the components disclosed herein may be implemented in hardware, software, or a combination of hardware and software (e.g., firmware). The software components may be in the form of a computer-readable program code stored in a computer-readable storage medium, such as memory, mass storage device, or removable storage device. For example, a computer-readable storage medium may comprise a computer-readable code for performing the function of a particular component. Likewise, a computer memory may be configured to include one or more components, which may then be executed by a processor. Components may be implemented separately in multiple modules or together in a single module.

Depending on its configuration, the computer 52 shown in the example of FIG. 3 may be employed as a desktop computer, a server computer, or an appliance, for example. The computer 52 may have less or more components to meet the needs of a particular application. As shown in FIG. 3, the computer 52 may include one or more processors 78, such as those from the Intel Corporation or Advanced Micro Devices, for example. The computer 52 may have one or more buses 82 coupling its various components. The computer may include one or more input devices 80 (e.g., keyboard, mouse), a computer-readable storage medium (CRSM) 84, a CRSM reader 86 (e.g., floppy drive, CD-ROM drive), a display monitor 88 (e.g., cathode ray tube, flat panel display), a network connection 90 (e.g., network adapter, modem) for coupling to a network, one or more data storage devices 92 (e.g., hard disk drive, optical drive, FLASH memory), a main memory 94 (e.g., RAM) and an interface 53 for communication with the components of the plasma generating system 10 as illustrated in FIG. 2. Software embodiments may be stored in a computer-readable storage medium 84 for reading into a data storage device 92 or the main memory 94 as illustrated in FIG. 3.

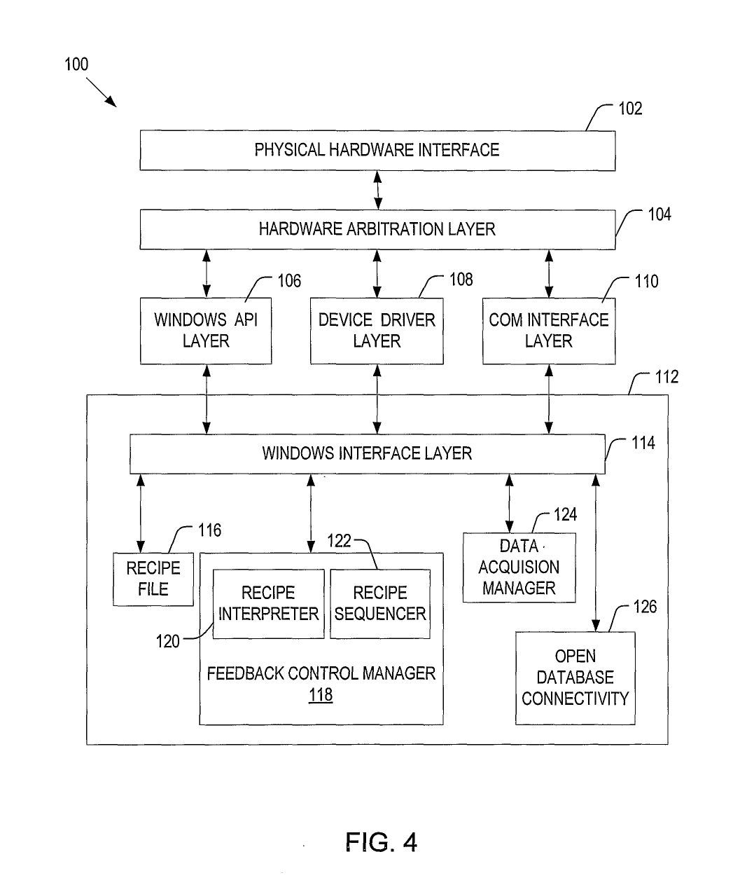

FIG. 4 is a schematic diagram of a software architecture 100 stored in the memory 94, where each component in the software architecture 100 relates to a feedback control/data acquisition in accordance with one embodiment of the present invention. As illustrated, the software architecture 100 may include: a physical hardware interface 102 configured to control hardware attached to the computer 52; a hardware arbitration layer 104 configured to provide the interface between hardware and a windows operating system; a windows application programming interface (API) layer 106 configured to provide the interface between windows applications and the hardware arbitration layer 104; a device driver layer 108 configured to provide the interface between the windows operating system and hardware devices attached to the computer 52; a common object model (COM) interface layer 110 configured to provide the interface between the windows application and the hardware device driver; and a system control/data acquisition manager 112. The system control/data acquisition manager 112 will be further explained in following sections.

In FIG. 4, the software architecture 100 is assumed to be compatible with a Microsoft Windows™ operating system. However, it should be apparent to those of ordinary skill in the art that the present invention may be practiced with any other type of operating system. Also, the physical hardware interface 102, the hardware arbitration layer 104, the windows API layer 106, the device driver layer 108 and the COM interface layer 110 may be implemented as default settings in an operating system.

Still referring to FIG. 4, the system control/data acquisition manager 112 may include: a windows interface layer 114 configured to communicate with the windows API layer 106, the device driver layer 108 and the COM interface layer 110 and to provide the interface to the windows applications; a recipe file 116 having at least one recipe that determines the operational conditions under which the measurement device 48 may perform the data acquisition; a feedback control manager 118 configured to operate the feedback control module 44 in conjunction with the pertinent components of the plasma generating system 10 that communicate with the feedback control module 44 (shown in FIG. 2), the feedback control manager 118 comprising a recipe interpreter 120 and a recipe sequencer 122; a data acquisition manager 124 configured to control the measurement device 48 for the data acquisition; and an Open DataBase Connectivity (ODBC) Application Programming Interface 126 configured to communicate with the data acquisition manager 124 and the data storage devices 92 (shown in FIG. 3) to store acquired data into a data storage medium. In one embodiment, the data acquisition manager 124 and the ODBC 126 may be implemented in another computer that may be coupled to the computer 52 for communication.

As mentioned, the feedback control manager 118 may be configured to operate the feedback control module 44 so that one or more operational objectives may be achieved. One objective may be maintaining an intended plasma condition, such as temperature, radiative emission or electron number density, during the operation of the plasma generating system 10. Another objective may be acquiring data under various plasma conditions in a systematic and a parametric manner, i.e., obtaining data while one or more values of parameters that determine the operational conditions are varied systematically. This mechanism may be especially important during the construction of a database for operational conditions of the plasma generating system 10. The database may include information related to plasma characteristics (e.g., plasma emission spectra) determined by a combination of various parameters, such as the power level of the power supply 16, the gas flow rate through the MFC valve 38, the slider position of the sliding short circuit 42, etc. Such parametric operations of a plasma generating system for database construction can be tedious, human error prone and, depending on the application of plasma, hazardous to operators, which may require the automation of data acquisition using the feedback control manager 118. The feedback control manager 118 may be configured to optimize the data acquisition so that, during automatic and parametric data acquisition processes, the measurement device 48 may skip data acquisition unless a stable plasma is established. Such optimization can be critical where each measurement generates data of a considerable size, such as a high resolution plasma image data.

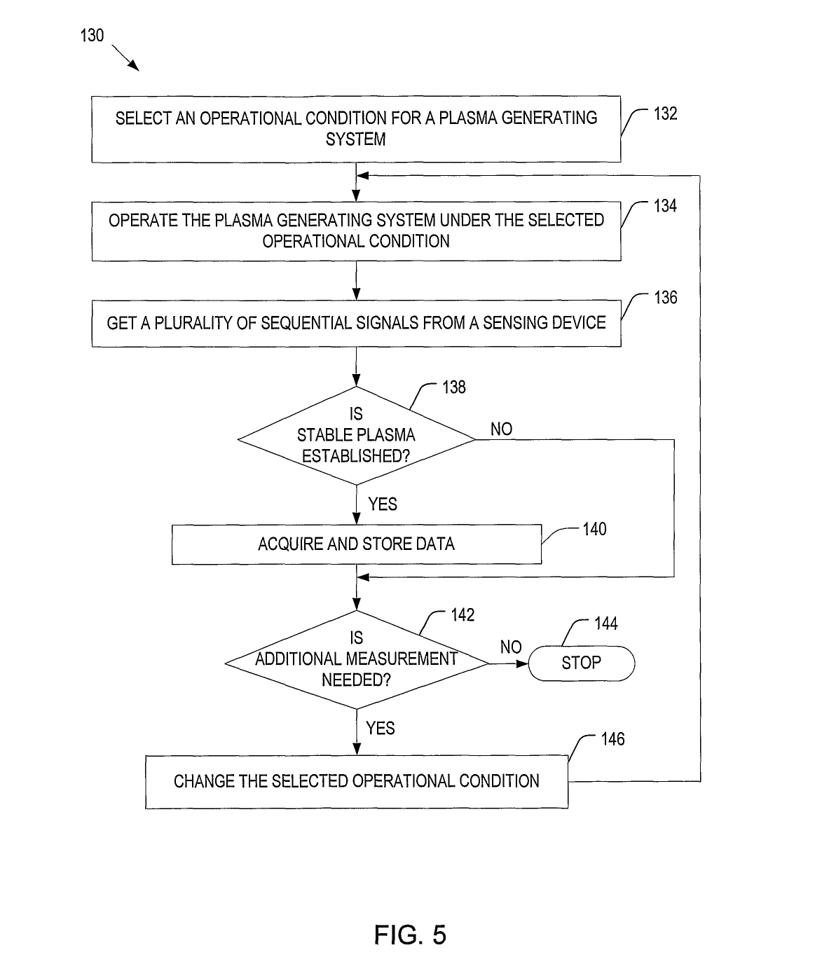

FIG. 5 shows a flowchart 130 illustrating exemplary steps of the feedback control manager 118 in conjunction with the plasma generating system 10 and the feedback control module/data acquisition system 11 in accordance with one embodiment of the present invention.

In step 132, the feedback control manager 118 may determine an operational condition for the plasma generating system 10 based on a selected recipe, where the determined operational condition may include a set of parameters comprising the power level of the power supply 16, the gas flow rate through the MFC valve 38, the state of the tuner 28 and the state of the sliding short circuit 42. Hereinafter, for purposes of the illustration and compactness of the disclosure, it is assumed that only two parameters, the power level of the power supply 16 and the gas flow rate through the MFC valve 38, may be varied to change the operational conditions while the other two parameters are fixed. However, it should be apparent to those of ordinary skill in the art that the other two parameters may be varied as well. Also, other plasma generating systems different from the system 10 may have different components communicating with their feedback control modules. In such systems, the types of control signals sent from their feedback control modules may be different than those from the feedback control module 44. However, it should also be apparent to those of ordinary skill in the art that the steps set forth in FIG. 5 may equally be applied to such systems regardless of the number of parameters included in an operational condition.

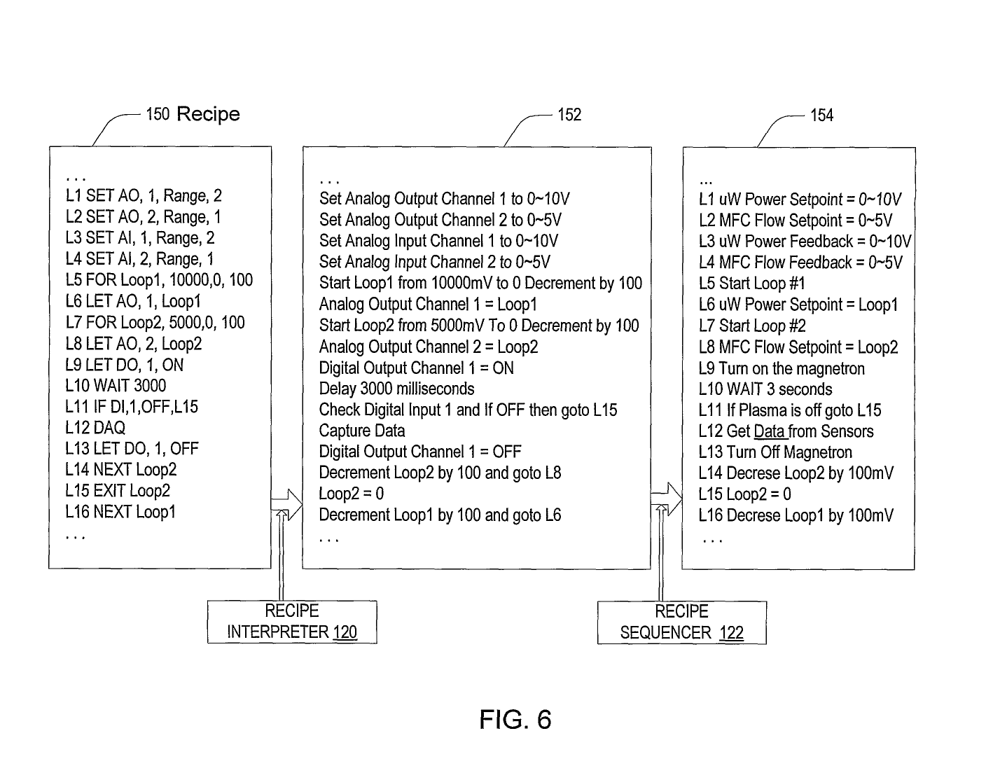

The selected recipe may be stored in a recipe file 116 and specify one or more operational conditions. Each recipe in the recipe file 116 may be written in, but not limited to, an extensible markup language (XML). A sample code segment of a recipe 150 is shown in FIG. 6. FIG. 6 is a block diagram illustrating steps for converting a recipe into a sequence of commands taken by the recipe interpreter 120 and the recipe sequencer 122 in step 132. As the recipe 150 is self-explanatory, a detailed description will not be given in the disclosure. The sample code is configured to provide a set of operational conditions for data acquisition by changing two parameters systematically; the power level of the power control 50 and the gas flow rate via the MFC valve 38, where the corresponding parameters are AO1 and AO2 in lines L1 and L2 of the recipe 150, respectively. The recipe interpreter 120 may translate the recipe 150 into an interpreted recipe 152 line-by-line. For example, “Range, 2” in line L1 of the recipe 150 maybe interpreted as a voltage range of 0-10 Volts for the power control 50. Subsequently, the recipe sequencer 122 may convert the interpreted recipe 152 into a sequenced recipe 154, where each line of the sequenced recipe 154 may correspond to a command (or equivalently, operating a corresponding component of the plasma generating system 10).

Referring now back to FIG. 5, in step 134, to operate the plasma generating system 10 under the determined operational condition, the feedback control manager 118 may command the feedback control module 44 to send appropriate signals to components of the plasma generating system 10. In addition to the signals for the power control 50 and the MFC valve 38, the feedback control manager 118 may also send other signals, such as “Turn on the magnetron” in L9 of the sequenced recipe 154 to turn on magnetron (or, equivalently, the microwave generator 14 in FIG. 1).

In step 136, a plurality of signals may be received from the sensor device 46 through lines 58a and 58b, and based on the received plurality of signals, the stability of the plasma 36 may be determined in a decision step 138. A first signal from the sensor device 46 can indicate whether a plasma ignition is successful. In case of success, the feedback control manager 118 may take one or more signals from the sensor device 46 during a preset time interval(s). The reset time interval may be specified in the recipe. For example, in the sequenced recipe 154, the preset time is set to 3 seconds in line L10. If the intensity variation of the plurality of signals is within an allowable range (or, equivalently a threshold), the plasma may be considered to be stable and the process proceeds to step 140 to acquire data. If the first signal indicates that the plasma ignition is unsuccessful and/or the intensity variation is greater than the allowable range, the process proceeds to step 142.

If the answer to the decision step 138 is YES, the feedback control manager 118 may communicate with the data acquisition manager 124 (shown in FIG. 4) so that the measurement device 48 may obtain data in step 140. In the same step, the data acquisition manager 124 may also communicate with the ODBC 126 to store the obtained data. Next, the feedback control manager 118 may determine whether any additional measurement is needed in step 142. Upon a negative answer to step 142, the process may stop at step 144. Otherwise, the power level of the power control 50 or the flow rate of the MFC valve 38 or both may be changed in step 146 and the process may proceed to step 134 for further measurements. In one embodiment, a set of measurements may be performed at a fixed gas flow rate while the power level of the power control 50 may be varied, preferably decreased by a preset percent thereof in step 146. Then, different sets of measurements may be performed at different gas flow rates, where the gas flow rate may be systematically decreased by a preset percent thereof to cover the entire matrix of test conditions, as illustrated in sequence recipe 154.

Upon a completion of the measurements under the operational conditions specified in the selected recipe, the feedback control manager 118 may automatically select another recipe stored in the recipe file 116 for further measurements in step 146 so that the entire recipes in the recipe file 116 are completed. Such an automated data acquisition process can prevent potential human errors and injuries by eliminating direct human involvement in the measurements and operation of the plasma generating system 10.

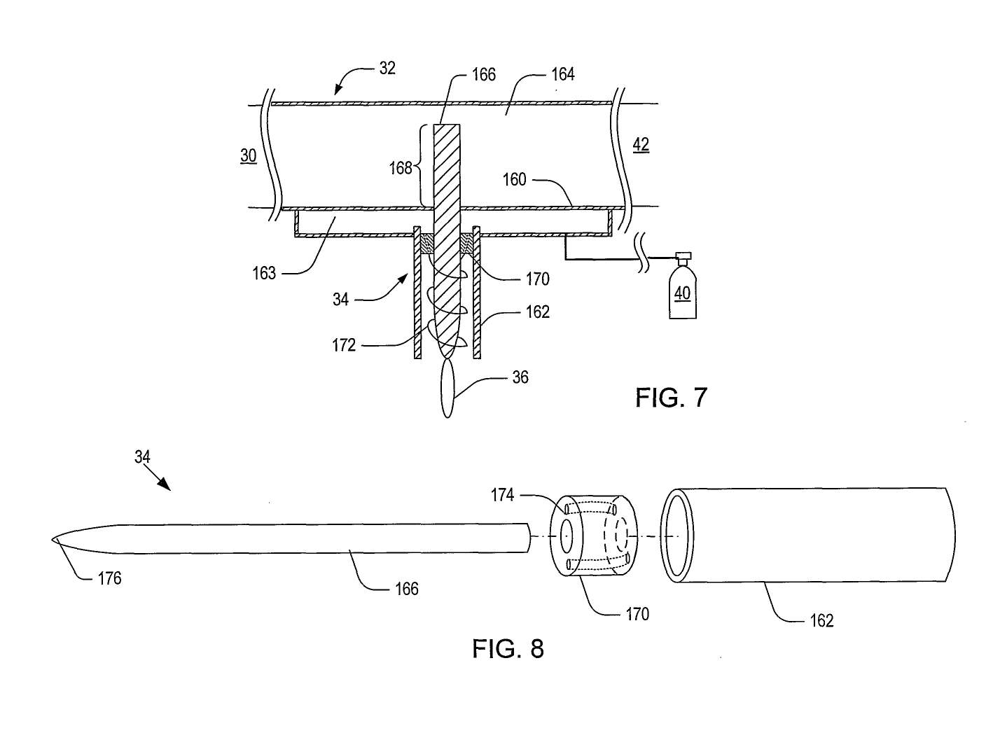

Referring back to FIG. 1, the feedback control module/data acquisition system 11 may be incorporated with any type of plasma generating system. FIG. 7 shows a partial cross-sectional view of an exemplary microwave cavity and nozzle shown in FIG. 1, taken along a plane parallel to the paper. As shown in FIG. 7, the microwave cavity 24 includes a wall 160 that forms a gas channel 163 for admitting gas from the gas tank 40; and a cavity 164 for containing the microwaves transmitted from the microwave generator 14. The nozzle 34 includes a gas flow tube 162 sealed with the cavity wall or the structure forming the gas channel 163 for receiving gas therefrom; a rod-shaped conductor 166 having a portion 168 disposed in the microwave cavity 24 for receiving microwaves from within the microwave cavity 164; and a vortex guide 170 disposed between the rod-shaped conductor 166 and the gas flow tube 162. The vortex guide 170 can be designed to securely hold the respective elements in place.

At least some parts of an outlet portion of the gas flow tube 162 can be made from conducting materials. The conducting materials used as part of the outer portion of the gas flow tube will act as a shield and it will improve plasma efficiencies. The part of the outlet portion using the conducting material can be disposed, for example, at the outlet edge of the gas flow tube.

FIG. 8 is an exploded view of the nozzle 34. As shown in FIG. 8, a rod-shaped conductor 166 and a gas flow tube 162 can engage the inner and outer perimeters of the vortex guide 170, respectively. A portion 168 of the rod-shaped conductor 166 acts as an antenna to collect microwaves from the microwave cavity 164 and focuses the collected microwaves to a tapered tip 176 to generate the plasma 36 using the gas flowing through the gas flow tube 162.

The rod-shaped conductor 166 may be made of any material that can conduct microwaves. The rod-shaped conductor 166 can be made out of copper, aluminum, platinum, gold, silver and other conducting materials. The term rod-shaped conductor is intended to cover conductors having various cross sections such as a circular, oval, elliptical, or an oblong cross section or combinations thereof. It is preferred that the rod-shaped conductor not have a cross section such that two portions thereof meet to form an angle (or sharp point) as the microwaves will concentrate in this area and decrease the efficiency of the device.

The gas flow tube 162 provides mechanical support for the overall nozzle 34 and may be made of any material that microwaves can pass through with very low loss of energy (substantially transparent to microwaves). Preferably, the material is a conventional dielectric material such as glass or quartz but it is not limited thereto.

The vortex guide 170 has at least one passage or a channel 174. The passage 174 (or passages) imparts a helical shaped flow direction around the rod-shaped conductor 166 to the gas flowing through the tube as shown in FIG. 7. A gas vortex flow path 172 allows for an increased length and stability of the plasma 36. It also allows for the conductor to be a shorter length than would otherwise be required for producing plasma. In one embodiment, the vortex guide 170 may be made of a ceramic material. The vortex guide 170 can be made out of any non-conducting material that can withstand exposure to high temperatures. Preferably, a high temperature plastic that is also a microwave transparent material is used for the vortex guide 170.

In FIG. 8, each through-pass hole or passage 174 is schematically illustrated as being angled to the longitudinal axis of the rod-shaped conductor and can be shaped so that a helical or spiral flow would be imparted to the gas flowing through the passage or passages. However, the passage or passages may have other geometric flow path shapes as long as the flow path causes a swirling flow around the rod-shaped conductor.

The three PCT applications previously referred to (PCT application entitled “Microwave Plasma Nozzle with Enhanced Plume Stability and Heating Efficiency” filed on Jul. 7, 2005, PCT Application entitled “System and Method for Controlling A Power Distribution Within a Microwave Cavity” filed Jul. 21, 2005 and PCT application entitled “Plasma Nozzle Array for Providing Uniform Scalable Microwave Plasma Generations filed Jul. 21, 2005) disclose variations of the nozzle 34 and microwave cavity 32 in detail. For simplicity, these variations are not described in the present document.

While the present invention has been described with reference to the specific embodiments thereof, it should be understood, of course, that the foregoing relates to preferred embodiments of the invention and that modifications may be made without departing from the spirit and the scope of the invention as set forth in the following claims.

What is claimed is:

1. A method for acquiring plasma data, comprising the steps of:
(a) selecting an operational condition for a plasma generation system;
(b) operating the plasma generation system under the operational condition selected in said step of selecting;
(c) determining whether a stable plasma is established using a sensing device;
(d) evaluating whether a stable plasma is determined in said step of determining, if so then said method includes the steps of:
acquiring data, and
storing the data obtained in said step of acquiring;
(e) determining whether an additional measurement is needed, wherein, if the additional measurement is not needed, said method further comprises the step of terminating data acquisition process;
(f) changing the operational condition selected in said step of selecting; and
(g) repeating said steps (b)-(f) for a new operational condition determined in said step of changing.

2. A method as defined in claim 1, wherein said step of determining whether a stable plasma is established comprises the steps of:
receiving a first signal from the sensing device;
determining, based on an intensity of the first signal, whether a plasma is ignited successfully, wherein, if unsuccessful, said method proceeds to said step (e);
receiving additional signals from the sensing device; and
determining whether the plasma is stable based on a fluctuation in intensity of the first signal and the additional signals.

3. A method as defined in claim 1, wherein the plasma generating system comprises a microwave generator, and wherein said step of changing the operational condition comprises:
changing a power level of the microwave generator by a preset percentage.

4. A method as defined in claim 1, wherein said step of operating the plasma generating system includes generating plasma by heating a gas flow, and wherein said step of changing the operational condition comprises the step of:
changing a gas flow rate by a preset percentage.

5. A method as defined in claim 1, further comprising the step of operating the sensing device responsive to a characteristic quantity of plasma.

6. A method as defined in claim 5, wherein the characteristic quantity is an amount of radiation emitted from the plasma, and wherein the sensing device is a photodiode, UV detector, phototransistor, photocell or photoconductive cell.

7. A method as defined in claim 1, wherein the characteristic quantity is a temperature of the plasma.

8. A computer readable medium including a program for carrying at least one sequence of instructions for optimally acquiring plasma data, wherein execution of the at least one sequence of instructions by the at least one processor causes the at least one processor to perform the steps of:
selecting an operational condition for a plasma generation system;
operating the plasma generation system under the operational condition selected in said step of selecting;
determining whether a stable plasma is established using a sensing device; and
if a stable plasma is established, then
acquiring data, and
storing the acquired data.

9. A computer readable medium as defined in claim 8, wherein execution of the at least one sequence of instructions by the at least one processor causes the at least one processor to perform the further steps of:
determining whether additional measurement is needed, wherein, if additional measurement is not needed, further comprising the step of terminating said steps of acquiring and storing;
changing an operational condition selected in said step of selecting an operational condition; and
repeating from said step of operating the plasma generation system to said step of changing.

10. A system for acquiring plasma data, the system comprising:
means for selecting an operational condition for a plasma generation system;
means for operating the plasma generation system under the operational condition;
means for determining whether a stable plasma is established using a sensing device; and
means for acquiring data and storing the acquired data.

11. A system as defined in claim 10, further comprising:
means for determining whether an additional measurement is needed and terminating a data acquisition process if the additional measurement is not needed;
means for changing to another operational condition; and
means for repeating operation of said means of operating the plasma generation system to said means for changing an operational condition.

12. A computer including a processor for running a computer-readable program code in a memory, said computer comprising:
a recipe file having at least one recipe that specifies at least one operational condition of a plasma generating system;
a feedback control manager structured and arranged to control said plasma generating system under the at least one operational condition, said feedback control manager comprising:
a recipe interpreter for interpreting the at least one recipe; and
a recipe sequencer for sequencing the recipe into at least one command to control said plasma generating system;
a data acquisition manager configured to acquire data if said plasma generating system generates a stable plasma under the at least one operational condition; and
an open database connectivity configured to store the data.

13. A system for acquiring plasma data, comprising:
a microwave generator for generating microwaves;
a power supply connected to said microwave generator for providing power thereto;
a microwave cavity having a wall forming a portion of a gas flow passage;
a waveguide operatively connected to said microwave cavity for transmitting microwaves thereto;
a coupler operatively connected to said waveguides;
a power meter, connected to the coupler, for measuring microwave fluxes;
an isolator, operatively connected to the waveguide, for dissipating microwaves reflected from said microwave cavity;
a gas flow control mechanism coupled to the gas flow passage of said microwave cavity for controlling a gas flow rate;
a nozzle operatively coupled to the gas flow passage of said microwave cavity and configured to generate plasma from a gas flow and microwaves received from said microwave cavity;
a sensing device configured to respond to a characteristic quantity of the plasma;
at least one measurement device configured to acquire data; and
a feedback control module connected to said power supply, said power meter, said sensing device, said at least one measurement device and said gas flow control, said feedback control module being configured to control said power supply, said at least one measurement device and said gas flow control and to receive at least one signal from said power meter and said sensing device.

14. A system as defined in claim 13, wherein said isolator includes:
a circulator operatively connected to said waveguide; and
a dummy load operatively connected to said circulator.

15. A system as defined in claim 13, further comprising:
a tuner coupled to said waveguide in proximity to said microwave cavity, wherein said feedback control module is coupled to and configured to control said tuner.

16. A system as defined in claim 13, further comprising:
a sliding short circuit operatively connected to said microwave cavity,
wherein said feedback control module is coupled to and configured to control said sliding short circuit.

17. A system as defined in claim 13, further comprising:
a gas flow tube for having a gas flow therethrough, said gas flow tube having an outlet portion including the nozzle and an inlet portion connected to said gas flow passage of said microwave cavity;
a rod-shaped conductor disposed in said gas flow tube, said rod-shaped conductor having a tapered tip disposed in proximity to said outlet portion of said gas flow tube, and wherein a portion of said rod-shaped conductor is disposed in said microwave cavity; and
a vortex guide disposed between said rod-shaped conductor and said gas flow tube, said vortex guide having at least one passage angled with respect to a longitudinal axis of said rod- shaped conductor for imparting a helical shaped flow direction around said rod-shaped conductor to a gas passing along said at least one passage.

18. A feedback control module for acquiring data of a plasma generated by a gas flow heated by microwaves, comprising:
a first field Input/Output coupled to a power control configured to control microwave generation;
a universal serial bus/general-purpose interface bus (UBS/GPIB) converter coupled to a power meter that is configured to measure fluxes of the microwaves;
a second field Input/Output coupled to a sensing device that is configured to generate a signal in response to a characteristic quantity of plasma;
a third field Input/Output coupled to a measurement device that is configured to acquire plasma data if a stable plasma is established;
a fourth field Input/Output coupled to a gas flow control; and
a computer having an interface coupled to said first, second, third and fourth field Input/Outputs and said USB/GPIB converter, and said interface comprising a plurality of interface components.

19. A feedback control module as defined in claim 18, further comprising:
a fifth field Input/Output coupled to a tuner that is configured to control reflection of microwaves and is coupled to said interface.

20. A feedback control module as defined in claim 19, further comprising:
a sixth field Input/Output coupled to a sliding short circuit and said interface.

21. A feedback control module as defined in claim 20, wherein said first, second, third, fourth, fifth and sixth field Input/Outputs are included in at least one field Input/Output module.

22. A method for acquiring plasma data, comprising the steps of:
(a) selecting an operational condition for a plasma generation system;
(b) operating the plasma generation system under the operational condition selected in said step of selecting; and
(c) determining whether a stable plasma is established using a sensing device; and
(d) evaluating whether a stable plasma is established in said step of determining, wherein in case of a successful establishment, further comprising the steps of acquiring data and storing the data obtained in said step of acquiring.

23. A method as defined in claim 22, further comprising the step of:
determining whether an additional measurement is needed, wherein, if the additional measurement is not needed, said method further comprises the step of terminating data acquisition process.

24. A method as defined in claim 22, further comprising the steps of:
changing the operational condition selected in said step of selecting; and repeating said steps (b)-(d) for a new operational condition determined in said step of changing.
